(19) United States
(12) Patent Application Publication
(10) Pub. No.: US 2013/0060260 A1
(43) Pub. Date: Mar. 7, 2013

(54) Multi-actuator array for the specific deformation of an implant

(71) Applicants: Sonja Dudziak, Bietigheim-Bissingen (DE); Thomas Rau, Hannover (DE); Thomas Lenarz, Hannover (DE); Omid Majdani, Hannover (DE)

(72) Inventors: Sonja Dudziak, Bietigheim-Bissingen (DE); Thomas Rau, Hannover (DE); Thomas Lenarz, Hannover (DE); Omid Majdani, Hannover (DE)

(21) Appl. No.: 13/547,402

(22) Filed: Jul. 12, 2012

(30) Foreign Application Priority Data
Jul. 15, 2011 (DE) 10 2011 107 778.6

(51) Int. Cl. A61B 19/00; A61F 2/18

(52) U.S. Cl. 606/130; 623/10

(57) ABSTRACT

An electronic control device assists in the insertion of an implant into the body of a patient, and allows the implant to be arbitrarily altered in shape by applying control signals. Patient-specific geometric body data from a body region into which the implant is to be introduced is input and stored. Computer-aided simulation of the insertion operation uses the patient-specific body data as a basis for simulating the insertion operation to produce actuation data which control the arbitrary alteration in the shape of the implant. The control device outputs the actuation data to the implant in the form of control signals on the basis of a sensed or calculated current introduction position of the implant according to the prior simulation.

### FIELD OF THE INVENTION

The aim is to insert electrode carriers into the spirally wound cochlea so as to preserve residual hearing. This is possible only when there are no or only minor forces applied to the surrounding tissue by the foreign body (electrode carrier). This is accomplished only when, throughout the entire course of the insertion, the electrode carrier follows an insertion path (trajectory) which is located centrally in the scala tympani. This is intended to avoid instances of contact and resultant contact forces with the surrounding tissue. At the end of the insertion, the electrode carrier is intended to apply itself gently to the inner wall and to come to rest permanently thereon.

### BACKGROUND

No electrode carrier which meets this demand has existed to date. The most protective electrode carriers to date have a particularly thin and flexible design. In this case, they are advanced along the outer wall and forced by the curved profile thereof into a likewise curved configuration. The result is constraining forces from the implant on the surrounding tissue and placement that is remote from the modiolus. The known alternative is implants produced in a precurved shape, which are initially held straight by stiffening structures (internal metal wires (stylet), rigid casings). When the stiffening structure has been removed, the electrode carriers return to the precurved shape and hence abut the inner wall at the end of the insertion.

Such deformation mechanisms have several fundamental shortcomings which have not been able to be resolved to date.

First, there is only one respective version size of the respective implant type available for all patients. Individual customization of the deformation response to suit the significantly varying anatomic (geometric) constraints of the inner ear is therefore not possible. This also allows no contactless/low-contact insertion, however, since the curvature response of the electrode carrier and the curvature of the cochlea cannot be attuned to one another to a sufficient degree.

Secondly, the known curvature mechanisms result in a basic discrepancy between the local curvature of the implant and the corresponding curvature of the inner ear at the same location, as a result of the course of time of the insertion. Thus, the electrode carrier returns directly and completely to its maximum curvature in the front region (see FIG. 1), which is already no longer being stiffened, even though the electrode is still in the only slightly curved region of the cochlea in this initial phase of the insertion process. This applies to a decreasing extent to the entire insertion process. Only at the maximum insertion depth has the curvature profile of the implant customized itself in the best possible manner to that of the inner ear—under the aforementioned restrictions that an average standard implant can map the individual anatomy only to a greatly restricted degree.

FIG. 2: Basic, local discrepancy between the curvature of the electrode carrier and the curvature of the cochlea. Whereas the electrode carrier returns directly and completely to its precurved shape, the basal regions of the cochlea still have an only slight curvature which increases toward the tip. Only following complete insertion has the curvature profile of the implant customized itself in the best possible manner to the cochlea. Instances of contact between the implant and the inner walls of the cochlea, which result in constraining forces on the functional tissue, are thereby inevitable.

This restriction applies equally to the use of other curvature mechanisms which are activated starting from the tip of the implant. In this context, it is also possible to cite the operating principle of a CI electrode carrier with three integrated shape memory actuators made of Nitinol that is cited by Hung Kha and Bernard Chen (see FIG. 3). Following activation by the body temperature, the curvature of the first actuator element will result in maximum flexure of the implant, even though the implant itself is still situated in the basal regions of the cochlea at this time in the insertion. Owing to this restriction, the authors also do not describe low-contact insertion at all as an objective. The only aim is to achieve a controlled final position in direct proximity to the basilar membrane by this means (“array which can be precisely located beneath the basilar membrane”). Since the documented level of development is also unable to achieve this, patient-individual optimized insertion by means of specific customization of the electrode curvature over the entire course of the insertion is also not the subject matter/objective of the development by Kha & Chen.

FIG. 3 shows a CI-electrode carrier with three integrated NiTi actuators which are each 6 mm in length. This is intended to achieve a final position in direct proximity to the basilar membrane in order to use the pump mechanism to apply medicaments in the closest proximity to the functional structures, as part of the basilar membrane.

FIG. 4 shows the phenomenon when actuator elements arranged in series—starting with the first—curve the implant into the intended final configuration.

Left: activation of the first 2 actuator elements 1, 2 results in the final (severe) curvature of the implant while the remainder of the implant 3 is still in the stretched configuration. Right: subsequent activation of the 3^{rd} actuator 3. At this time in the insertion, the implant is still situated in initial regions of the cochlear convolutions, however, which have only moderate curvature.

It therefore a particular challenge to the invention to use specific activation of the actuator elements to achieve such curvature of the implant as can be customized to the respective local curvature (with a local differentiation) of the cochlea over the entire course of the insertion i.e. with temporal differentiation.

### DESCRIPTION OF THE DRAWINGS

FIG. 1 presents side views of an implant where the electrode carrier is curved in its front region.

FIG. 2 illustrates discrepancies between the curvature of an electrode carrier and the curvature of the cochlea of a person.

FIG. 3 shows an electrode carrier with three integrated shape memory actuators.

FIG. 4 presents left and right panels illustrating bending of the implant where the left panel shows schematically actuation of the first two actuators of FIG. 3, and the right panel shows schematically actuation of the last actuator of FIG. 3.

FIG. 5 shows an exemplary multiactuator array for an implant according to the invention.

FIG. 6 shows circuitry implementations for externally driving the activation of the individual elements where a separated contact connection is made for each actuator element.

FIG. 7 shows a circuit implementation where a common ground line is used.

FIG. 8 shows an exemplary circuit implementation where three actuator elements have different geometric properties at the top and different material properties at the bottom.

FIG. 9 shows an arrangement with a pair of actuator elements being used to achieve a less severely pronounced curvature first (left panel) and a final curvature on later activation of one of the actuators (right panel).

FIG. 10 schematically shows actuator elements arranged in parallel.

FIG. 11 shows three actuator elements arranged as layers where each layer may have a different phase conversion temperature and each layer has a different longitudinal extent.

FIG. 12 shows a plurality of actuators arranged in series where different actuators in the series may have different phase conversion temperatures.

FIG. 13 shows electrical contact connections to each of a plurality of actuators in series.

FIG. 14 shows a multilayered actuator with electrical contact being made on one side of the multilayered structure.

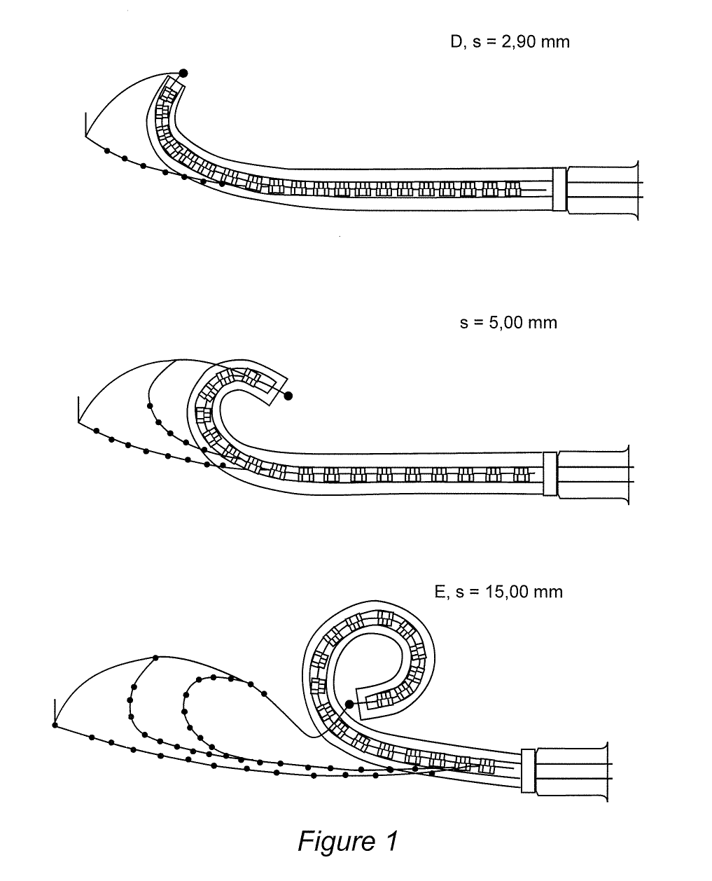

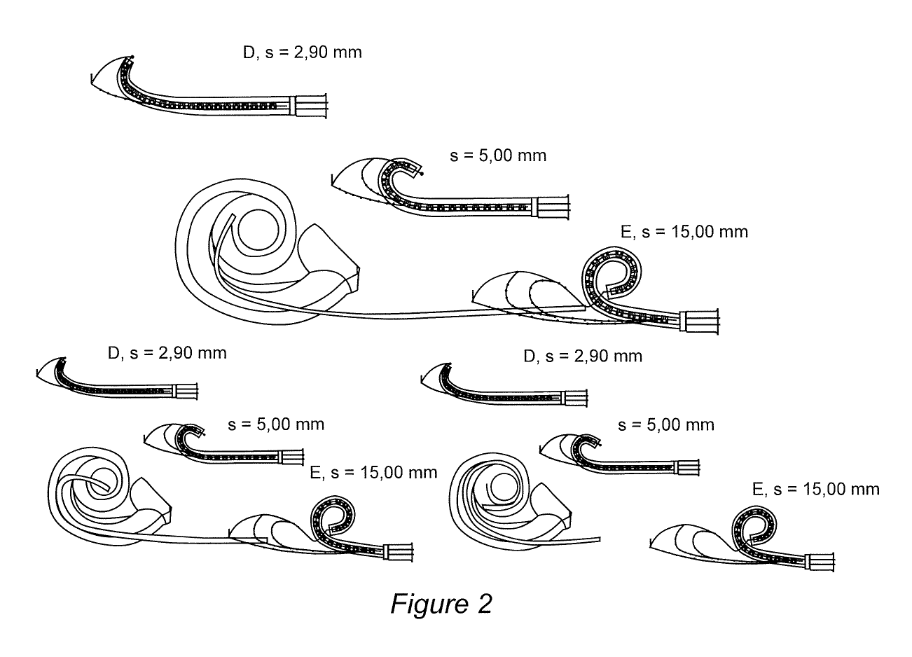

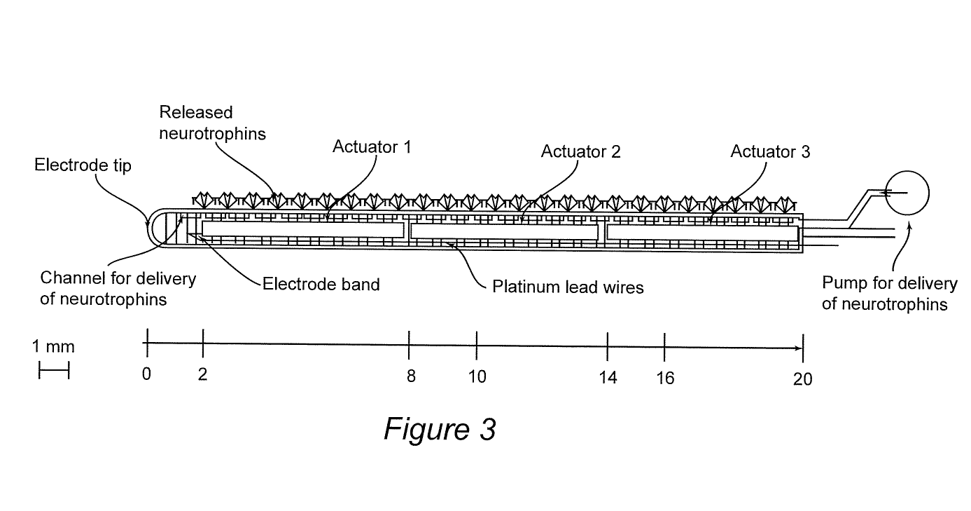

### DETAILED DESCRIPTION

### Solution to the Technical Problem by the Invention

### Multi-Actuator Design:

The problem of the lack of individualization of the insertion process for CI electrodes is solved by a specifically switchable actuator array which is integrated into the electrode carrier. This allows the temporally and/or locally differentiated curvature of the implant to be implemented for the purpose of customization to the curvature profile of the cochlea. Said implant consists of either a segmented actuator element or a plurality of individual actuators. These are preferably produced by means of a microlaser sintering method from Nitinol or are implemented by appropriately shaped and taught wires made from Nitinol. Alternatives are “melt spun ribbons” (thin strips which are manufactured by pouring out the Nitinol melt onto a cooled copper plate) or Nitinol structures deposited using thin layer methods. Besides Nitinol, there are naturally also other shape memory materials which are suitable for such use, such as shape memory polymers. The latter even have the further advantage that they do not increase the overall rigidity of the implant. This allows the patient-individual insertion to be achieved by specific activation—with economical production of a standard implant (or a product range reduced to a limited number of “convection sizes”)—without needing to produce a specific implant for each patient.

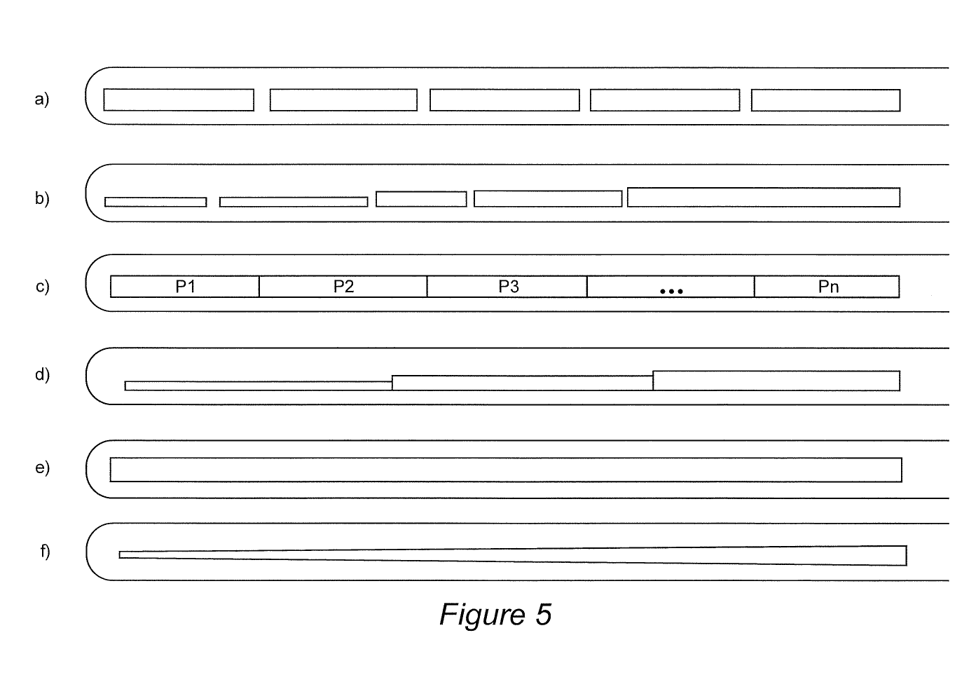

FIG. 5 shows an implementation of the multiactuator array by n individual actuator elements (a), these being able to be of locally different design in terms of size, shape and material properties, (b) or by an actuator element which varies on a segment-by-segment basis, in which material parameters vary from segment to segment (e.g. transition temperature, c) or form and shape parameters are varied (d) or else instead of a large number of n actuator elements or actuator segments the actuator is produced with graduated material parameters (e) or geometry parameters (f).

### Temporally and Locally Differentiated Curvature

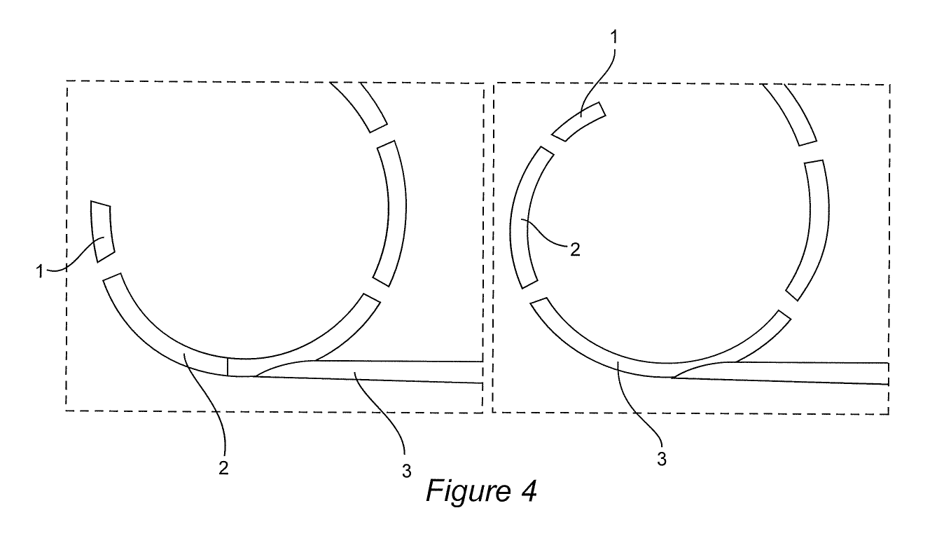

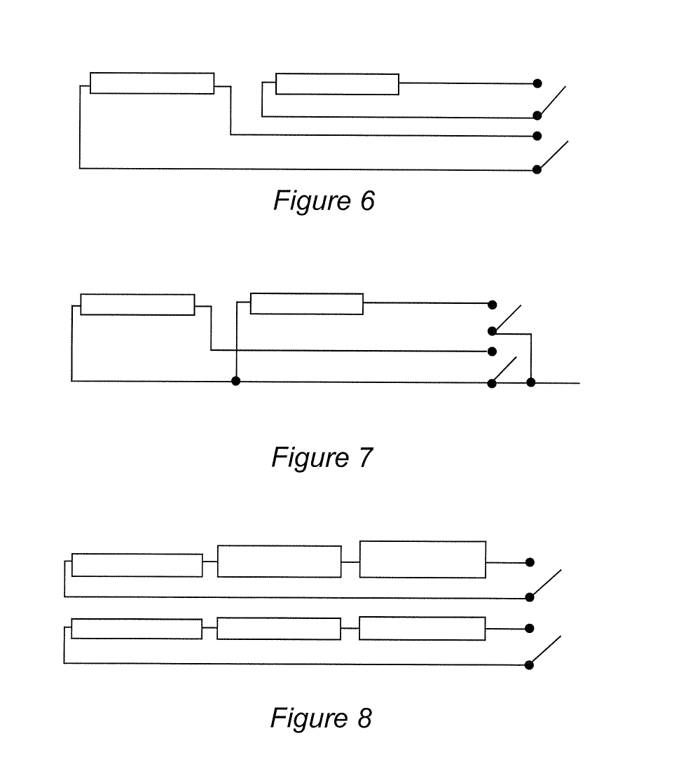

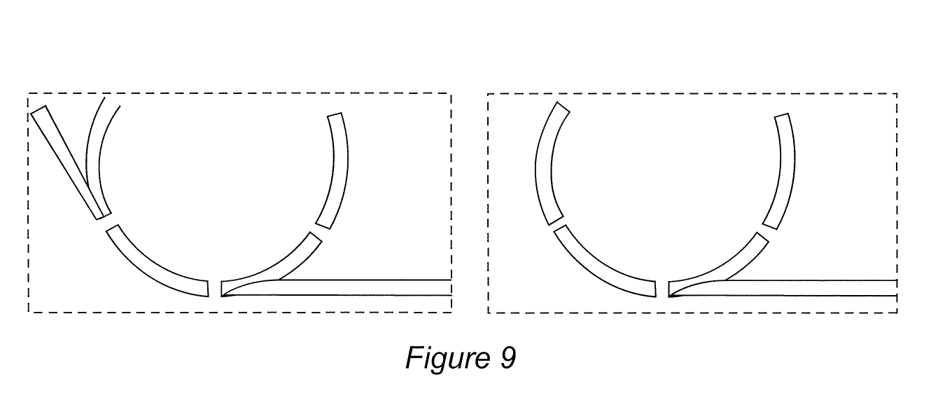

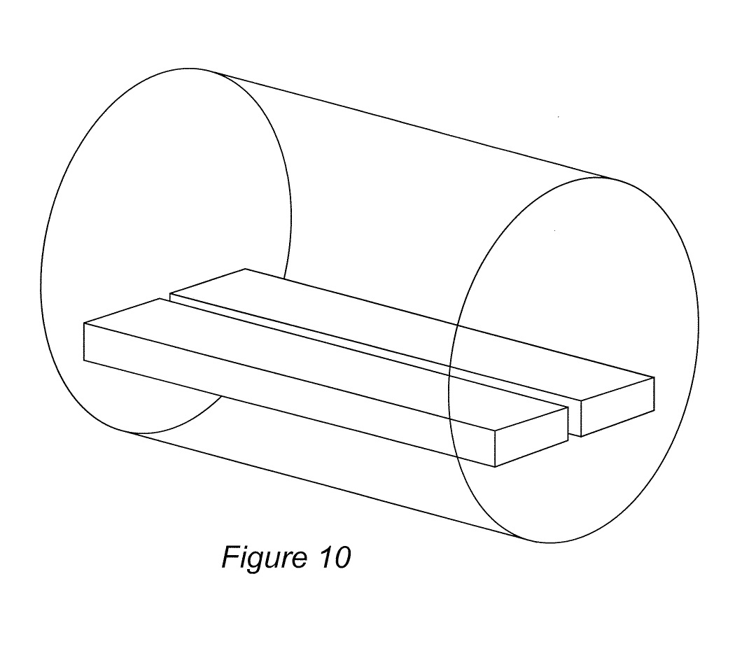

In order to overcome the problem of conventional curvature mechanisms that is outlined in FIG. 4, which achieve their maximum curvature too quickly (in the sense of the course of time of the insertion) starting from the tip, said maximum curvature therefore corresponding to that of the local (“local” means the respective location within the cochlea at which the implant is situated during a particular instant in the insertion) curvature of the inner ear, the following solution options are available as part of the invention:
1.) Temporally delayed transition of the actuator element from the stretched to the fully curved state when heated by the body temperature
   This temporally delayed transition can be coordinated with the advancement speed such that better customization of the curvature response from the implant to suit the low-contact/contactless insertion path is possible. Temporal delays arise in the case of activation by the body temperature as a result of the necessary conduction of heat from the surrounding intracochlear fluid, which is body temperature, through the silicone sheath to the SM material (SM=shape memory) of the actuator elements. Secondly, it requires time for the actuator element to be completely heated through, which means that only subsequently is the maximum deformation obtained. Thirdly, the course of time can be influenced by the choice of material parameters (particularly transition temperature). At relatively low transition temperatures, the SM effect is triggered by the body temperature more readily and more quickly than at relatively high transition temperatures. Transition temperatures above the body temperature are also possible, since the SM effect is also activated in that case, but it is triggered only with a delay and the respective actuator element does not return completely to the final shape. By customizing the material parameters, it is therefore possible to implement a temporally and locally differentiated curvature profile. Fourthly, it is possible to influence the speed and manifestation of the shape memory effect. Silicone sheaths, contact electrodes and contact-connection cables as stiffening structures counteract the actuating movement of the SM actuators, which means that the final configuration that appears is a tension equilibrium in the implant.
   The external driving of the advancement speed therefore allows what are known as effects/mechanisms to be used for individualizing the insertion process.
2.) Temporally delayed transition of the actuator element from the stretched to the completely curved state with external activation, e.g. by resistance heating (should Nitinol not be used as the shape memory material, it is also possible for other “triggering” effects to be used in a similar manner. For example, activation by photo effects or electrical or magnetic fields).
   The effects/mechanisms described under 1.) can be utilized in a similar manner with external activation in order to implement a temporally and locally differentiated deformation response. The simplest case of external activation is by resistance heating, when a current flows through the SM actuators. This can be implemented in different ways:
   a) Separate contact-connection of each actuator element
      In this case, two respective lines for the closed circuit are routed to the actuator element. External driving can thus be used to implement the activation of the individual element (see FIG. 6).
   b) Common ground line for reducing the number of wires (reducing the rigidity)
      In this case, a respective line is routed to the actuator element. However, the circuit is closed by means of a common ground line (see FIG. 7).
   c) Common contact-connection with graduated actuator elements or actuator segments
      If just the choice of the material parameters (e.g. transition temperature) implements temporal discretization of the activation of the SM effect (see 1.) or the measure of completeness of the effect, it is also possible for the resistance heating to be effected by a common circuit. In this case, c1) an identical voltage/current may be applied, which means that the application of heat to the actuator elements/actuator segments is the same, but these are activated thereby in temporally and locally differentiated fashion on account of the different material parameters. Alternatively c2) the flow of current can be increased in the course of time, which means that the application of heat in each actuator element/segment increases in the course of time. Actuator elements with a relatively low transmission temperature, for example, are thereby activated more readily than those with relatively high transition temperatures. The same applies to actuator elements with relatively large or relatively small volumes to be heated, different cross-sectional areas (hence a different resistance) and different geometry or material parameters, for example.
      FIG. 8 shows 3 actuator elements with different geometric properties at the top and varying material parameters at the bottom, by way of example.
   d) BUS system for driving and activating.
      A further reduction in the number of supply lines is possible when each contact electrode has an upstream “address decoder” (control element) which uses the addressing to evaluate whether the downstream flow of current is intended for “its own” actuator element and accordingly enables said actuator element. As a result, appropriately high clock frequencies, a plurality of actuator elements can be heated proportionately or completely on a highly discrete-time basis. The currently available methods of microelectronics easily allow the manufacture of such electronic circuit elements in the necessary order of magnitude. In addition, it will be possible to integrate temperate sensors, so that each local control element can also return information about the state of the actuator to the extracochlear central control element.
3.) Paired or grouped connection of actuator elements in series
   Besides the cited effects, it is also possible for the individualization of the temporally and locally resolved curvature response to be amplified or modified by further effects of the external driving as well. One option is the grouped connection of actuator elements arranged in succession. If, as in the schematic figure below, for example, the actuator element located more basally is activated first, it is possible to achieve a geometric intermediate configuration in which the more moderate curvature better corresponds to the curvature profile of the cochlea in basal sections. Only in the further course of the insertion is it possible to activate the actuator element(s) located more apically in order to transfer the implant to its final geometry/curvature.
   FIG. 9 shows a series circuit comprising a pair of actuator elements in order to achieve an intermediate configuration with less severely pronounced curvature first when the more basal actuator in activated (profile on the left) and to achieve the final curvature only upon later activation of the more apical actuator (profile on the right).
4.) Paired or grouped connection of actuator elements in parallel
   A comparable effect can be achieved when actuator elements are connected in parallel. With appropriate design, it is possible for the actuating force of an individual actuator element initially not to suffice to achieve the final curvature of the electrode carrier, which means that an intermediate configuration with a less pronounced curvature profile appears, which curvature profile better corresponds to basal sections of the cochlea (see FIG. 10).
   Temporal effects as described in 1.) and 2.) allow continuous, temporally and locally differentiated changes in the electrode configuration (curvature profile) to be implemented in combination with 3.) or 4.) in order to map the wound profile of the individual inner ear to the best possible extent. This makes it possible to avoid contact forces with the surrounding tissue.
   The intraoperative implementation of such a patient-specific insertion strategy is assisted by the use of the presented automated insertion tool with programmable electrode advancement. The patient-specific insertion strategy can be determined by using simulations.
      Further features are
   the active switching of the actuators, which is not effected on the basis of the body temperature,
   the overall concept consisting of prior measurement of the cochlea, possible manufacture of patient-specific multi-actuator implants, monitoring during the insertion process and controlled, active switching of the multi-actuator implants,
   a laser sintering method can be used to adjust the switching temperature sufficiently precisely for active switching of the actuators to be effected at temperatures which do not result in damage to the hearing, but at the same time are sufficiently above the body temperature, as a result of which the actuators do not switch without active heating,
   the overall implant (actuators plus electrode carriers), which is designed such that electrical driving is sufficiently electrically insulated from the cochlea and that the voltages required for switching will be low enough for the insulation to be sufficient.

One embodiment of the invention relates to an implant for insertion into the body of a patient, particularly a cochlear implant, wherein the implant can be arbitrarily altered in shape by applying control signals, wherein the implant has a multiplicity of actuators which are distributed over the physical extent of the implant, which can be arbitrarily actuated by control signals individually or in groups, and which, when actuated, bring about an alteration in the shape of the implant. By way of example, the actuators can be actuated by electrical signals, by light signals or by magnetic fields.

One embodiment of the invention relates to a method for manufacturing an implant of the type described previously, wherein the shape alteration element is machined by using a microlaser sintering method.

In this case, the implant may have, in particular, a multi-actuator implant with electrodes arranged thereon which are used to stimulate the cochlea. In this way, the multi-actuator implant can also simultaneously be used as an electrode carrier for the cochlear implant, for example.

One embodiment of the invention relates to an implant of the type described previously, wherein the implant has a sensor device which is set up to sense the distance between the implant and surrounding body tissue of the patient and/or to sense a contact force between the implant and the surrounding body tissue of the patient.

One embodiment of the invention relates to an electronic control device for a medical system which is set up to assist the insertion of an implant into the body of a patient, wherein the implant can be arbitrarily altered in shape by applying control signals.

One embodiment of the invention relates to an implant of the type described previously, wherein the implant is set up for connection to an actuation data output means of an electronic control device according to one of claims 1 to 7.

One embodiment of the invention relates to a computer program having program code means, set up to control the flow control means of the electronic control device of the type described previously, when the computer program is executed on a computer, such that control signals are output on the basis of a sensed or calculated current introduction position of the implant according to the prior simulation, which control signals are based on previously determined actuation data which are set up to control the arbitrary alteration in the shape of the implant.

One embodiment of the invention relates to a computer program of the type described previously, wherein the computer program is set up to perform the computer-aided simulation of the insertion operation for the implant into the body on the basis of the patient-specific body data.

One embodiment of the invention relates to a computer program of the type described previously, wherein the computer program is stored on a machine-readable medium.

As mentioned, the implant according to the invention can be used to bring about specific activation of the individual actuator elements. This makes it possible to achieve a curvature for the implant which is such that it can be customized over the entire course of the insertion, i.e. with temporal differentiation, and of the respective local curvature of the cochlea, i.e. with local differentiation.

The shape memory actuators can be manufactured by using a laser additive production process, for example. It is advantageous to reduce the speed of travel of the scanner mirrors between the exposure units of a laser coating device in order to reduce vibrations in the scanner mirrors at the path starts of coating paths, for example. It is also advantageous to avoid speeds of travel of over 150 mm per second, in order likewise to prevent or at least reduce vibrations in the scanner mirrors during exposure. It is furthermore advantageous to increase the delay time prior to an acceleration by a scanner mirror. This allows the scanner mirror to stop vibrating. It is additionally advantageous, in order to reduce the phase conversion temperature of a shape memory actuator, i.e. the activation temperature thereof, to manufacture the shape memory actuator at a lower scan speed than when a higher phase conversion temperature is desired. To achieve a higher phase conversion temperature at constant scan speed, it is possible to increase the laser power, for example. An increase or reduction in the phase conversion temperature following the laser melting process is dependent on the nickel/titanium ratio of the powder material. A powder with a relatively high nickel content results in a reduced phase conversion temperature, at low laser power and constant scan speed. A powder with a relatively high titanium content results in a constant phase conversion temperature at low laser power and constant scan speed.

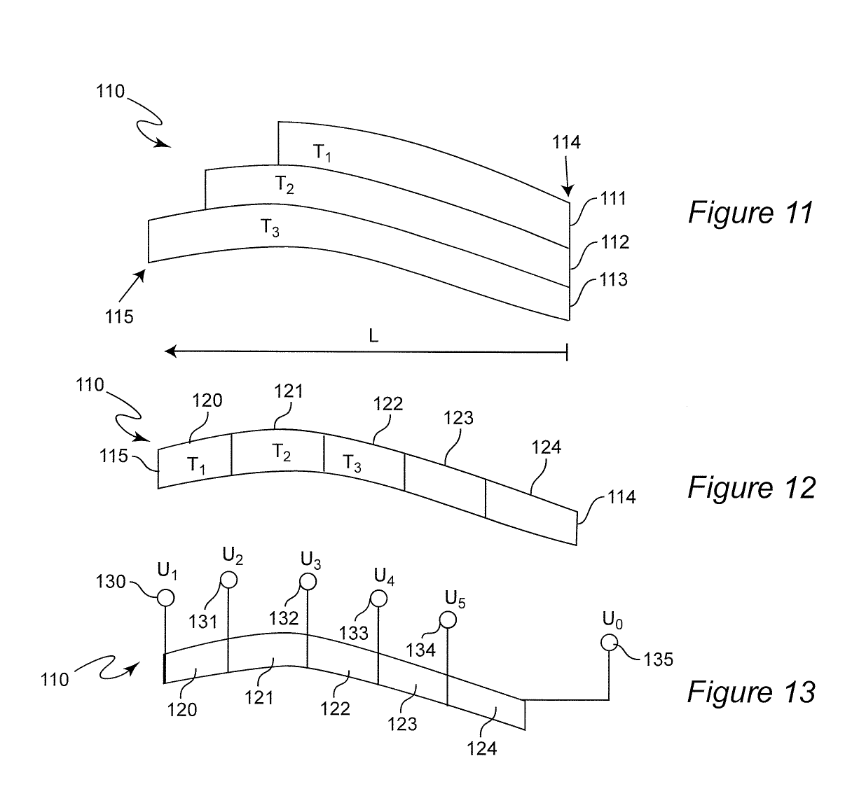

FIGS. 11 to 14 show further embodiments of the actuators in the form of a multi-actuator implant which can be used as an electrode carrier for a cochlear implant. In this case, that end of the electrode carrier 110 which is ahead when the cochlear implant is introduced—said end also being called the introduction side—is denoted by the reference symbol 115 in each case. The opposite end of the electrode carrier 110 is denoted by the reference symbol 114. The electrode carrier 110 therefore extends in the longitudinal extent in a direction L, as shown in FIG. 11, from the side 114 to the introduction side 115.

As can be seen in FIG. 11, the electrode carrier may be constructed from three actuators 111, 112, 113 arranged above one another in layers, it also being possible for more than the three layers shown to be provided. The actuators 111, 112, 113 are electrically activatable and each change their shape at different phase conversion temperatures T1, T2, T3 as a result of the electrical activation. The actuators 111, 112, 113 have longitudinal extents of different magnitude in the direction L of the electrode carrier 110, with the actuators 111, 112, 113 ending at different distances from the introduction side 115. This results in a certain gradation for the actuators from the introduction side 115.

FIG. 12 shows an embodiment of an electrode carrier 110 in which a plurality of actuators 120, 121, 122, 123, 124 are arranged in succession in the direction of the longitudinal extent L of the electrode carrier, i.e. extend in succession from the side 114 to the introduction side 115. It is again possible for different phase conversion temperatures for the individual actuators to be provided.

FIG. 13 shows one option for the electrical contact-connection of the actuators 120, 121, 122, 123, 124 by virtue of electrical connections 130, 131, 132, 133, 134, 135 being arranged at each of the border points between two actuators. An actuator is actuated by applying an actuation voltage to respective contacts of the electrode carrier.

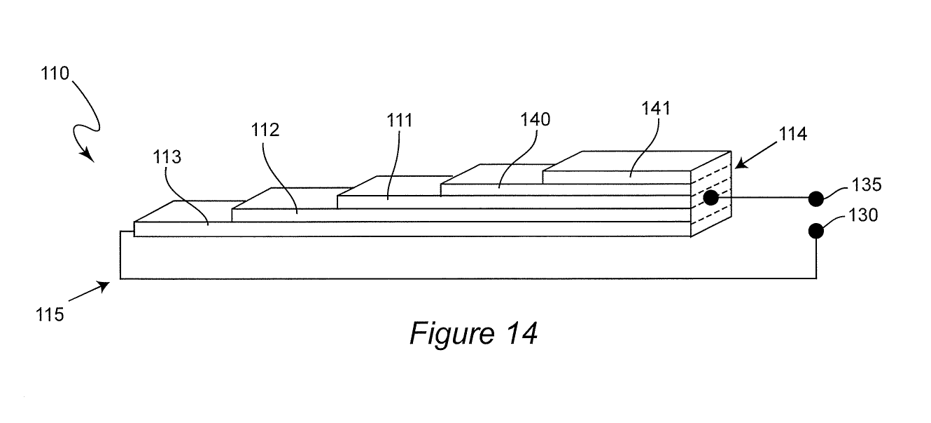

FIG. 14 shows a similar embodiment of the electrode carrier 110 to FIG. 11, but with five actuator layers 113, 112, 111, 140, 141. Electrical connections 130, 135 are provided on the side 114 and on the introduction side 115, respectively. When an electrical voltage is applied, the individual actuators are heated on account of the electrical current flowing through the electrode carrier. In this case, the actuator with the lowest phase conversion temperature is actuated first, followed by the others in gradual succession.

Patent claims:

1. Electronic control device for a medical system which is set up to assist the insertion of an implant into the body of a patient, wherein the implant can be arbitrarily altered in shape by applying control signals,
a) wherein the control device has at least one input and storage means which is set up to input and store patient-specific geometric body data from that body region into which the implant is to be introduced,
b) wherein the control device has at least one simulation means for the computer-aided simulation of the insertion operation for the implant into the body, wherein the simulation means is set up to take the patient-specific body data as a basis for simulating the insertion operation for the implant into the body in order to produce actuation data which are set up to control the arbitrary alteration in the shape of the implant,
c) wherein the control device has at least one actuation data output means for outputting the actuation data to the implant in the form of control signals to which actuation data output means the implant can be connected, and
d) wherein the control device has at least one flow control means which is set up to output the control signals on the basis of a sensed or calculated current introduction position for the implant according to the prior simulation.

2. Electronic control device according to claim 1, wherein the simulation means is set up to automatically avoid contact with the body when simulating the insertion operation for the implant into the body and to produce the actuation data therefrom.

3. Electronic control device according to claim 1, wherein the flow control means is set up to output the control signals in sync with an advancement movement of the implant.

4. Electronic control device according to claim 1, wherein the control device has at least one sensing means to which at least one position sensor can be connected which is set up to report the current introduction position of the implant to the sensing means.

5. Electronic control device according to claim 1, wherein the simulation means is set up to produce control data when simulating the insertion operation for the implant into the body, which control data are set up to control a robot arm which is used for the automated introduction of the implant into the body, wherein the control device has at least one control data output means for outputting the control data to a robot arm, to which control data output means a robot arm can be connected.

6. Electronic control device according to claim 5, wherein the flow control means is set up to calculate the current introduction position of the implant from the control data for controlling the robot arm.

7. Electronic control device according to claim 5, wherein the simulation means is set up to produce introduction position data when simulating the insertion operation for the implant into the body, which introduction position data indicate the current introduction position of the implant in relation to the control data for controlling the robot arm when the robot arm is introducing the implant into the body.

8. Medical system which is set up to assist the insertion of an implant into the body of a patient wherein the implant can be arbitrarily altered in shape by applying control signals, wherein the medical system has at least one control device according to claim 1.

9. Medical system according to claim 8, wherein the medical system has at least one robot arm which is set up for the automated introduction of an implant into the body of a patient wherein the robot arm is controlled by virtue of its being connected to the control data output means of the control device.

10. Medical system according to claim 8, wherein the medical system has at least one position sensor which is set up to report the current introduction position of the implant to the sensing means of the control device, wherein the position sensor is connected to the sensing means of the control device.

11. Implant for insertion into the body of a patient, particularly a cochlea implant, wherein the implant can be arbitrarily altered in shape by applying control signals, wherein the implant has a multiplicity of actuators which are distributed over the physical extent of the implant, which can be arbitrarily actuated by control signals individually or in groups, and which, when actuated, bring about an alteration in the shape of the implant.

12. Implant according to claim 11, wherein the actuators can be electrically actuated via one or more electrical lines which are routed along the implant and which are electrically insulated from the surroundings and from one another.

13. Implant according to claim 11, wherein one, a plurality of or all actuator(s) has/have an electrically actuatable heating element or is/are itself/themselves in the form of an electrically actuatable heating element.

14. Implant according to claim 11, wherein the implant has a shape alteration element, of single-part or multi-part design, made of at least one shape memory material and the actuators are at least thermally coupled to the shape alteration element or the actuators consist of the shape memory material

15. Implant according to claim 14, wherein the shape alteration element is of layered design comprising a plurality of layers of the shape memory material which overlap at least over a portion of the longitudinal extent of the implant.

16. Implant according to claim 15, wherein the individual layers of the shape memory material have lengths which differ gradually in the direction of the longitudinal extent of the implant.

17. Implant according to claim 16, wherein the layers of the shape memory material end gradually at different distances from the introduction side of the implant.

18. Implant according to claim 15, wherein the individual layers of the shape alteration element have different activation temperatures for the shape memory material.

19. Implant according to claim 11, wherein the shape alteration element has a plurality of electrical connection contacts which are arranged in succession over the longitudinal extent of the implant and which are set up to drive individual actuators of the shape alteration element arbitrarily individually.

20. Implant according to claim 11, wherein one, a plurality of or all actuators has/have an insulating casing, particularly a silicone casing.
